(19) United States
(12) Patent Application Publication
(10) Pub. No.: US 2002/0035083 A1
(43) Pub. Date: Mar. 21, 2002

(54) CRF2 ligands in combination therapy

(76) Inventor: Siew Peng Ho, Chadds Ford, PA (US)

Correspondence Address: BRISTOL-MYERS SQUIBB PHARMA COMPANY, PATENT DEPARTMENT, P.O. BOX 4000, PRINCETON, NJ, 08543-4000, US

(21) Appl. No.: 09/908,825

(22) Filed: Jul. 19, 2001

Related U.S. Application Data
(60) Provisional application No. 60/219,391, filed on Jul. 19, 2000.

(51) Int. Cl. A61K 48/00; C07H 21/04

(52) U.S. Cl. 514/44; 536/23.5

(57) ABSTRACT

This invention relates to antisense oligonucleotides directed against the mRNA of the corticotropin releasing factor subtype-2 (CRF₂) receptor which substantially reduce expression of CRF₂ receptors in the rodent brain and the use of antisense oligonucleotides in in vivo CNS studies of gene function and to treat a wide range of psychiatric disorders including anxiety, obsessive-compulsive disorder, panic disorders, post-traumatic stress disorder, phobias and depression.

### FIELD OF THE INVENTION

The invention is directed to a pharmaceutical composition comprising a CRF₁ receptor ligand and a CRF₂ receptor ligand, or pharmaceutically acceptable salts or prodrugs thereof; and to a method of treating a disorder associated with CRF₁, and CRF₂ receptor activity, comprising administering to a patient in need thereof a therapeutically effective amount of a CRF₁ receptor ligand and a CRF₂ receptor ligand, or pharmaceutically acceptable salts or prodrugs thereof, wherein CRF receptor ligands of this invention are agonists or antagonists of the CRF receptors. In addition to the pharmaceutical target of the invention being the CRF receptors, this invention is also directed to pharmaceutical agents which target CRF₁ and CRF₂ receptor mRNA.

### BACKGROUND OF THE INVENTION

Extensive studies have established the importance of corticotropin releasing factor (CRF) in controlling the pituitary-adrenocortical system and in mediating the behavioral, autonomic and immune responses to stress. Hence, this peptide is thought to be involved in the pathophysiology of affective disorders. Presently, two 7-transmembrane receptors, CRF₁ and CRF₂, have been identified which mediate the effects of CRF. Both receptors are widely expressed in brain although there is little significant overlap between the areas of highest expression of the two receptor sub-types. CRF-overexpressing transgenic mice have been reported to exhibit an increase in anxiogenic (anxiety-producing) behavior (Stenzel-Poore et al., Overproduction of corticotropin-releasing factor in transgenic mice: A genetic model of anxiogenic behavior. J. Neuroscience 14, 2579-2584, 1995). Of particular importance is the question of whether these anxiogenic responses are mediated through CRF action on CRF₁ receptors, CRF₂ receptors or both.

Corticotropin-releasing factor (CRF) antagonists are mentioned in U.S. Pat. Nos. 4,605,642, 5,874,227, 5,962,479, 5,063,245, 5,861,398 and 6,083,948, which are incorporated herein by reference in their entirety. Several published patent applications also disclose corticotropin releasing factor antagonist compounds, among these are DuPont Merck PCT application US 94/11050, Pfizer WO 95/33750, Pfizer WO 95/34563, Pfizer WO 95/33727 and U.S. Pat. No. 5,424,311. Diseases considered treatable with CRF antagonists are discussed in U.S. Pat. No. 5,063,245 and Pharm. Rev., 43: 425-473 (1991).

A role for CRF has also been postulated in the etiology and pathophysiology of Alzheimer's disease, Parkinson's disease, Huntington's disease, anorexia nervosa, progressive supranuclear palsy and amyotrophic lateral sclerosis as they relate to the dysfunction of CRF neurons in the central nervous system [for review see E. B. De Souza, Hosp. Practice 23:59 (1988); G. N. Smagin, L. A. Howell, D. H. Ryan, E. B. De Souza and R. B. S. Harris Neuroreport 9, 1601-1601, 1998; and J. Pharmacol. Exp. Therap., 293, 700-806, 2000;]. U.S. Pat. No. 6,051,578, which is incorporated herein by reference in its entirety, discloses (CRF) receptor antagonist which are useful in the treatment and prevention of head trauma, spinal cord trauma, ischemic neuronal damage (e.g., cerebral ischemia such as cerebral hippocampal ischemia), excitotoxic neuronal damage, epilepsy, stroke, stress induced immune dysfunctions, phobias, muscular spasms, Parkinson's disease, Huntington's disease, urinary incontinence, senile dementia of the Alzheimer's type, multiinfarct dementia, amyotrophic lateral sclerosis, chemical dependencies and addictions (e.g., dependencies on alcohol, cocaine, heroin, benzodiazepines, or other drugs), and hypoglycemia.

U.S. Pat. No. 6,001,807, which is incorporated herein by reference in its entirety, discloses (CRF) receptor antagonist which are useful in the treatment and prevention of emesis. The anti-emetic activity of the CRF-antagonists is indicated by experiments performed for example as described by Ueno et al, Life Sciences 41: 513-518 (1987); and Rudd et al., British Journal of Pharmacology 119: 931-936 (1996).

Also, a number of publications disclose CRF₁ receptor antagonists, for example Chen et al., J. Med. Chem. 39: 4358-4360 (1996); Whitten et al., J. Med. Chem. 39: 4354-4357 (1996); Chen et al., J. Med. Chem. 40(11) 1749-1754 (1997); Lundkvist et al., Eur. J. Pharmacoloy. 309, 198-200, 1996; and Mansbach et al., Eur. J. Pharmacoloy. 323, 21-26, 1997, which are incorporated herein by reference in their entirety. More specifically the the CRF₁ receptor ligand DPC904 is disclosed in Gilligan et al., BioOrganic Medicinal Chem. 8, 181-189, 2000, which is incorporated herein by reference in its entirety.

Also, CRF₂ receptor ligands, for example sauvagine, urocortin and other CRF₂ peptides, are disclosed in Ho et al., Mol. Brain Res. 6, 11, 1998; J. Spiess et al., Trends Endocrinology and Metabolism 9, 140-145, 1998 Molecular Properties of the CRF Receptor; and D. P. Behan et al., Mol. Psychiatry 1, 265-277, 1996, which is incorporated herein by reference in its entirety.

While blockade of CRF₁ receptors by selective antagonists has been shown to produce anxiolytic (anxiety-reducing) and anti-depressant effects in animals, the function of CRF₂ receptors is less well studied. In situ hybridization and receptor autoradiography experiments show the receptor to be localized primarily in the limbic and hypothalamic brain regions, suggesting a role in mediating the anxiogenic and anorexic effects of CRF. Recently, a CRF₂-selective antagonist (Anti-Sauvagine-30) has been identified (Gulyas J. et al. (1995) Proc. Natl. Acad. Sci. USA 92, 10575-579). Furthermore, Astressin, a peptide having dual CRF1 and CRF2 activity has been identified (Ruhmann, A., Bonk, I., Lin, C. R., Rosenfeld, M. G. & Spiess, J. (1998) Proc. Natl. Acad. Sci. USA 95, 15264-15269). In the absence of specific agonists or antagonists to this receptor, antisense suppression of CRF₂ receptor expression may provide evidence for the role of the receptor in normal physiology.

Antisense oligonucleotides are short oligonucleotides (typically from about 15 to about 25 nucleotides in length) which are designed to be complementary to a portion of an mRNA molecule of interest. Hybridization of an antisense oligonucleotide to its mRNA target site through Watson-Crick base-pairing initiates a cascade of events which terminate in oligonucleotide-directed degradation of the targeted mRNA molecule. A direct consequence of this mRNA degradation is the suppression of synthesis of the encoded protein. Studies done in the presence of significantly reduced levels of the targeted protein may reveal its function. In the absence of small molecule ligands (as is the case with the CRF₂ receptor), antisense oligonucleotides can be extremely useful tools for protein functional studies. In addition, they can be used to distinguish between closely related members of a family of proteins (such as CRF₁ and CRF₂) in ways which are often not possible with small molecule ligands.

The design and selection of potent antisense sequences is not a trivial exercise. Antisense oligonucleotides vary widely and unpredictably in their activity because their mRNA targets have significant secondary and tertiary structure which render larger portions of an mRNA molecule inaccessible to hybridization. Only 20-35% of antisense sequences have significant inhibitory activity (50% or more). Using a molecular technique we developed (Ho et al., Potent antisense oligonucleotides to the human multidrug resistance-1 mRNA are rationally selected by mapping RNA-accessible sites with oligonucleotide libraries. Nucl. Acids Res. 24, 1901-1907, 1996; Ho et al., Mapping of RNA accessible sites for antisense experiments with oligonucleotide libraries. Nature Biotech. 16, 59-63, 1998), multiple accessible regions in the CRF₂ receptor mRNA were identified. Antisense oligonucleotides directed against these accessible sites inhibited the binding of ¹²⁵I-sauvagine to CRF₂ receptors in vivo by at least 50%.

Two antisense studies examining the function of CRF₂ receptors have been reported. Both studies failed to find evidence for involvement of the CRF₂ receptor in mediating the anxiogenic effects of CRF. However, in one study (Heinrichs et al., Corticotropin-releasing factor CRF₁ but not CRF₂, receptors mediate anxiogenic-like behavior. Reg. Peptides 71, 15-21, 1997), CRF₂ receptors were reduced by only 15-20%, and the oligonucleotides used produced toxic side effects (significant weight loss) which could have confounded the behavioral experiments. Little detail was provided in the second report (Montkowski et al., Biol. Psychiatry 39, 566, 1996; and Liebsch, G., Landgraf, R., Engelmann, M., Lorscher, P. & Holsboer, F. (1999) J. Psychiatric Res. 33, 153-163.

However, in a study using CRF₂ antisense oligonucleotides which are described in International Patent Application No. PCT/US00/0819 and U.S. patent application Ser. No. 09/481,981, which are incorporated herein in their entirety, we have discovered that suppression of CRF₂ receptor expression produces anxiolytic effects in animals.

Furthermore, we have discovered that when the CRF₂ receptor antisense oligonucleotide is co-administered with a CRF₁, receptor ligand, the anxiolytic effect is greatly enhanced.

### SUMMARY OF THE INVENTION

This invention relates to a method of treating a disorder associated with CRF₁ and CRF₂ receptor activity, comprising administering to a patient in need thereof a therapeutically effective amount of a CRF₁ receptor ligand and a CRF₂ receptor ligand, or pharmaceutically acceptable salts or prodrugs thereof.

In one embodiment, the present invention provides a method of treating a disorder associated with CRF₁ and CRF₂ receptor activity, comprising administering to a patient in need thereof a therapeutically effective amount of a CRF₁ receptor ligand and a CRF₂ receptor ligand, or pharmaceutically acceptable salts or prodrugs thereof, wherein the CRF₁ ligand receptor is agonistic of the CRF₁ receptor.

In another embodiment, the present invention provides a method of treating a disorder associated with CRF₁ and CRF₂ receptor activity, comprising administering to a patient in need thereof a therapeutically effective amount of a CRF₁ receptor ligand and a CRF₂ receptor ligand, or pharmaceutically acceptable salts or prodrugs thereof, wherein the CRF₁ ligand receptor is antagonistic of the CRF₁ receptor.

In yet another embodiment, the present invention provides a method of treating a disorder associated with CRF₁ and CRF₂ receptor activity, comprising administering to a patient in need thereof a therapeutically effective amount of a CRF₁ receptor ligand and a CRF₂ receptor ligand, or pharmaceutically acceptable salts or prodrugs thereof, wherein the CRF₂ ligand receptor is agonistic of the CRF₂ receptor.

In still another embodiment, the present invention provides a method of treating a disorder associated with CRF₁ and CRF₂ receptor activity, comprising administering to a patient in need thereof a therapeutically effective amount of a CRF₁ receptor ligand and a CRF₂ receptor ligand, or pharmaceutically acceptable salts or prodrugs thereof, wherein the CRF₂ ligand receptor is antagonistic of the CRF₂ receptor.

In a further embodiment, the present invention provides a method of treating a disorder associated with CRF₁ and CRF₂ receptor activity, comprising administering to a patient in need thereof a therapeutically effective amount of a CRF₁ receptor ligand and a CRF2 receptor antisense oligonucleotide, or pharmaceutically acceptable salts or prodrugs thereof, wherein the CRF2 receptor antisense oligonucleotide is an antisense oligonucleotide composed of chimeric oligonucleotides wherein between 10-70% of the 2′-deoxyribonucleotide phosphorothioate residues are replaced with modified nucleotide residues.

In yet a further embodiment, the present invention provides a method of treating a disorder associated with CRF₁ and CRF₂ receptor activity, comprising administering to a patient in need thereof a therapeutically effective amount of a CRF₁ receptor ligand and a CRF2 receptor antisense oligonucleotide, or pharmaceutically acceptable salts or prodrugs thereof, wherein the CRF2 receptor antisense oligonucleotide is an antisense oligonucleotide composed of chimeric oligonucleotides wherein between 10-70% of the 2′-deoxyribonucleotide phosphorothioate residues are replaced with modified nucleotide residues selected from the following group: 2′-methoxyribonucleotide phosphodiesters, 2′-methoxy-ethoxyribonucleotide phosphodiesters, 2′-fluoro-ribonucleotide phosphodiesters, 5-(1-propynyl)cytosine phosphorothioate, 5-(1-propynyl)uracil phosphorothioate, 5-methyl cytosine phosphorothioate, 2′-deoxyribonucleotide-N3′-P5′phosphoramidate, and polyamide nucleic acids, and locked nucleic acids having the formula:

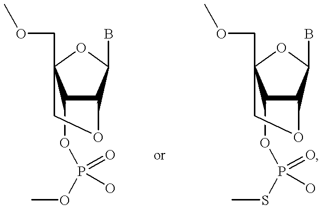

wherein B is a purine or pyimidine base.

In still a further embodiment, the present invention provides a method of treating a disorder associated with CRF₁ and CRF₂ receptor activity, comprising administering to a patient in need thereof a therapeutically effective amount of a CRF₁ receptor ligand and a CRF2 receptor antisense oligonucleotide, or pharmaceutically acceptable salts or prodrugs thereof, wherein the CRF2 receptor antisense oligonucleotide is an antisense oligonucleotides composed of chimeric oligonucleotides wherein between 10-70% of the 2′-deoxyribonucleotide phosphorothioate residues are replaced with modified nucleotide residues, wherein the oligonucleotide is from about 15 to about 25 nucleotides in length.

In another embodiment, the present invention provides a method of treating a disorder associated with CRF₁ and CRF₂ receptor activity, comprising administering to a patient in need thereof a therapeutically effective amount of a CRF₁ receptor ligand and a CRF₂ receptor antisense oligonucleotide, or pharmaceutically acceptable salts or prodrugs thereof, wherein the CRF₂ receptor antisense oligonucleotide is an antisense oligonucleotides composed of chimeric oligonucleotides, wherein between 60-70% of the 2′-deoxyribonucleotide phosphorothioate residues of the antisense oligonucleotides are replaced with modified nucleotide residues.

In yet another embodiment the present invention provides a method of treating a disorder associated with CRF₁ and CRF₂ receptor activity, comprising administering to a patient in need thereof a therapeutically effective amount of a CRF₁ receptor ligand and a CRF2 receptor antisense oligonucleotide, or pharmaceutically acceptable salts or prodrugs thereof, wherein the CRF2 receptor antisense oligonucleotide is an antisense oligonucleotides comprising the following sequences:

(a) TGT ACG TGT TGC GCA AGA GG;

(b) GGT GGG CGA TGT GGG AAT G;

(c) GGA TGA AGG TGG TGA TGA GG; and

(d) TGA CGC AGC GGC ACC AGA CC.

In still another embodiment, the present invention provides a method of treating a disorder associated with CRF₁ and CRF₂ receptor activity, comprising administering to a patient in need thereof a therapeutically effective amount of a CRF₁ receptor ligand and a CRF2 receptor antisense oligonucleotide, or pharmaceutically acceptable salts or prodrugs thereof, wherein the disorder is a psychiatric disorder.

In a further embodiment, the present invention provides a method of treating a psychiatric disorder associated with CRF₁ and CRF₂ receptor activity, comprising administering to a patient in need thereof a therapeutically effective amount of a CRF₁ receptor ligand and a CRF₂ receptor ligand, or pharmaceutically acceptable salts or prodrugs thereof, wherein the psychiatric disorder is selected from the group consisting of anxiety, obsessive-compulsive disorder, panic disorders, post-traumatic stress disorder, phobias, anorexia nervosa, and depression.

In another embodiment, the present invention provides a method of treating a disorder associated with CRF₁ and CRF₂ receptor activity, comprising administering to a patient in need thereof a therapeutically effective amount of a CRF₁ receptor ligand and a CRF₂ receptor ligand, or pharmaceutically acceptable salts or prodrugs thereof, wherein the disorder is selected from the group consisting of head trauma, spinal cord trauma, ischemic neuronal damage (e.g., cerebral ischemia such as cerebral hippocampal ischemia), excitotoxic neuronal damage, epilepsy, stroke, stress induced immune dysfunctions, phobias, muscular spasms, Parkinson's disease, Huntington's disease, urinary incontinence, senile dementia of the Alzheimer's type, multiinfarct dementia, amyotrophic lateral sclerosis, chemical dependencies and addictions (e.g., dependencies on alcohol, cocaine, heroin, benzodiazepines, or other drugs), and hypoglycemia.

In another embodiment, the present invention provides a method of treating a disorder associated with CRF₁ and CRF₂ receptor activity, comprising administering to a patient in need thereof a therapeutically effective amount of a CRF₁ receptor ligand and a CRF₂ receptor ligand, or pharmaceutically acceptable salts or prodrugs thereof, wherein administering the CRF₁ receptor ligand and the CRF₂ receptor ligand is concurrent.

In still another embodiment, the present invention provides a method of treating a disorder associated with CRF₁ and CRF₂ receptor activity, comprising administering to a patient in need thereof a therapeutically effective amount of a CRF₁ receptor ligand and a CRF₂ receptor ligand, or pharmaceutically acceptable salts or prodrugs thereof, wherein administering the CRF₁ receptor ligand and the CRF₂ receptor ligand is sequential.

In yet another embodiment, the present invention provides a method of treating a disorder associated with CRF₁ and CRF₂ receptor activity, comprising contacting an effective amount of a CRF₁ receptor ligand and a CRF₂ receptor ligand with a composition containing CRF₁ receptor and CRF₂ receptor.

In yet another embodiment, the present invention provides a method of treating a disorder associated with CRF₁ and CRF₂ receptor activity, comprising contacting an effective amount of a CRF₁ receptor ligand and a CRF2 receptor antisense oligonucleotide with a composition containing CRF₁ receptor, wherein the CRF2 receptor antisense oligonucleotide is an antisense oligonucleotides composed of chimeric oligonucleotides wherein between 10-70% of the 2′-deoxyribonucleotide phosphorothioate residues are replaced with modified nucleotide residues.

In a further embodiment the present invention relates to treating a disorder associated with CRF₂ receptor activity, comprising contacting an effective amount of a CRF₂ receptor ligand with a composition containing CRF₂ receptor.

In yet a further embodiment, the present invention provides a pharmaceutical composition comprising a CRF₁ receptor ligand and a CRF₂ receptor ligand, or pharmaceutically acceptable salts or prodrugs thereof, and a pharmaceutical carrier.

In still a further embodiment, the present invention provides a pharmaceutical kit for treating or preventing a disorder associated with CRF₁ and CRF₂ receptor activity, said kit comprising a plurality of separate containers, wherein at least one of said containers contains a CRF₁ receptor ligand, or a pharmaceutically acceptable salt or prodrug thereof, and at least another of said containers contains a CRF₂ receptor ligand, or pharmaceutically acceptable salts or prodrugs thereof, and said containers optionally contain a pharmaceutical carrier.

In another embodiment, the present invention provides a pharmaceutical kit for treating or preventing a disorder associated with CRF₁ and CRF₂ receptor activity, said kit comprising a plurality of separate containers, wherein at least one of said containers contains a CRF₁ receptor ligand, or a pharmaceutically acceptable salt or prodrug thereof, and at least another of said containers contains a CRF2 receptor antisense oligonucleotide, or pharmaceutically acceptable salts or prodrugs thereof, and said containers optionally contain a pharmaceutical carrier.

In still another embodiment, the invention provides a compound having CRF₁ receptor ligand activity and a CRF₂ receptor ligand activity for use in the treatment of psychiatric disorders.

In yet another embodiment, the present invention provides antisense oligonucleotides directed against the mRNA of the CRF₂ receptor which substantially reduce expression of CRF₂ receptors in the rodent brain. Suppression of CRF₂ receptor function using these oligonucleotides produced significant anxiolytic (anxiety-reducing) effects in animals. These data provide the first functional evidence that CRF₂ receptors play an important role in mediating the anxiogenic (anxiety-producing) effects of corticotropin releasing factor. Furthermore, the data demonstrate the potential of CRF₂ receptor antagonists, including small molecules, to be effective in the treatment of a wide range of psychiatric disorders including anxiety, obsessive-compulsive disorder, panic disorders, post-traumatic stress disorder, phobias and depression.

In a further embodiment, the present invention provides a method of treating psychiatric disorders including, but not limited to, anxiety, obsessive-compulsive disorder, panic disorders, post-traumatic stress disorder, phobias and depression in a patient, by administering to the patient requiring such treatment a therapeutically effective amount of a pharmaceutical composition comprising antisense oligonucleotides comprised of chimeric oligonucleotides where 10-70% of the 2′-deoxyribonucleotide phosphorothioate residues are replaced with modified nucleotide residues.

In yet a further embodiment, the invention provides a method of screening compounds to determine activity for the treatment of psychiatric disorders including, but not limited to, anxiety, obsessive-compulsive disorder, panic disorders, post-traumatic stress disorder, phobias and depression.

In still a further embodiment the invention provides antisense oligonucleotides composed of chimeric oligonucleotides wherein between 10-70% of the 2′-deoxyribonucleotide phosphorothioate residues are replaced with modified nucleotide residues.

### BRIEF DESCRIPTION OF THE DRAWINGS

Certain embodiments of the invention have been chosen for purposes of illustration and description, but are not intended in any way to restrict the scope of the invention. These embodiments of the invention are shown in the accompanying drawings described below.

FIG. 1a: Schematic for antisense sequence selection.

FIG. 1b: Identity of chimeric, semi-random oligonucleotide libraries.

FIG. 2a: Structure of most commonly used nucleotide analogs in antisense studies; the phosphorothioate variation produces CNS toxic effects.

FIG. 2b: Structure of modified oligonucleotide analogs which maintain potency but eliminate toxicity when incorporated into oligonucleotides for CNS applications.

FIG. 2c: One of several possible configurations for chimeric oligonucleotides.

FIG. 3a: Effect of antisense oligonucleotides on freezing behavior in rats.

FIG. 3b: Inhibition of ¹²⁵I-sauvagine binding in the lateral septum of antisense treated rats in the freezing assay.

FIG. 4a: Effect of antisense treatment on rodent behavior in the elevated plus maze.

FIG. 4b: Inhibition of ¹²⁵I-sauvagine binding in the lateral septum of antisense treated rats in the elevated plus maze assay.

FIG. 5: Effect of antisauvagine-30 on freezing behavior in rats.

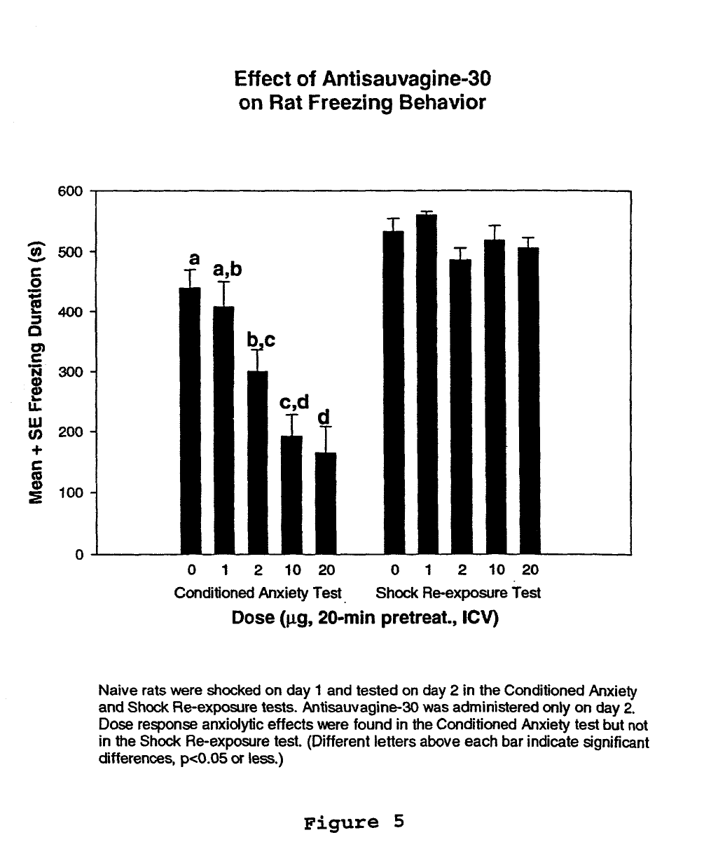

FIG. 6: Effect of combining a CRF₂ receptor antisense olignucleotide with a CRF1 antagonist on freezing behavior in rats.

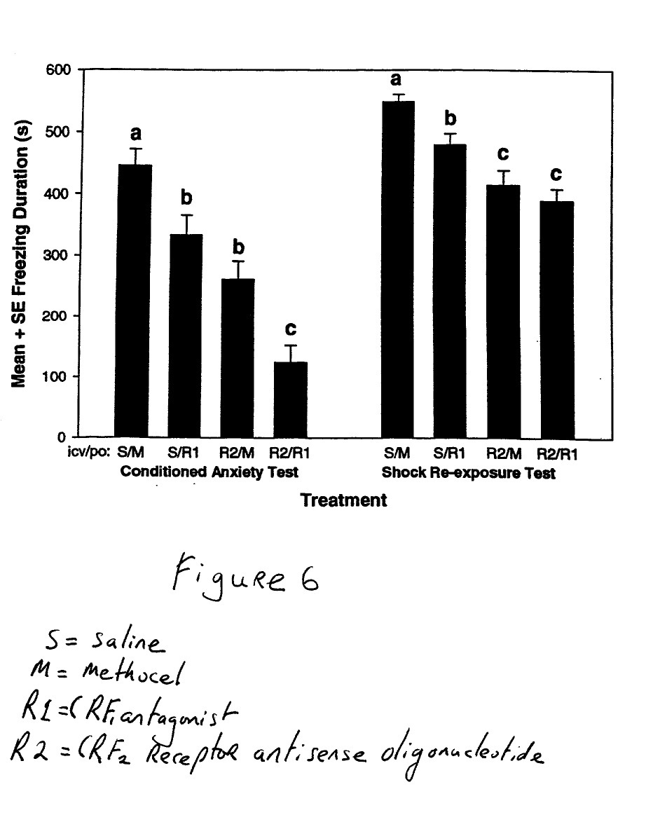

### DETAILED DESCRIPTION OF THE INVENTION

Not every antisense oligonucleotide is capable of potent inhibitory activity, and oligonucleotides targeting the CRF₂ receptor mRNA are no exception to that rule. Identification of active antisense sequences is one of the more important parameters which determine the success of antisense experiments. The factors which influence the potency of antisense sequences are complex and poorly understood; consequently only 20-35% of antisense oligonucleotides tested are sufficiently active to produce a 50% inhibitory effect on targeted protein synthesis.

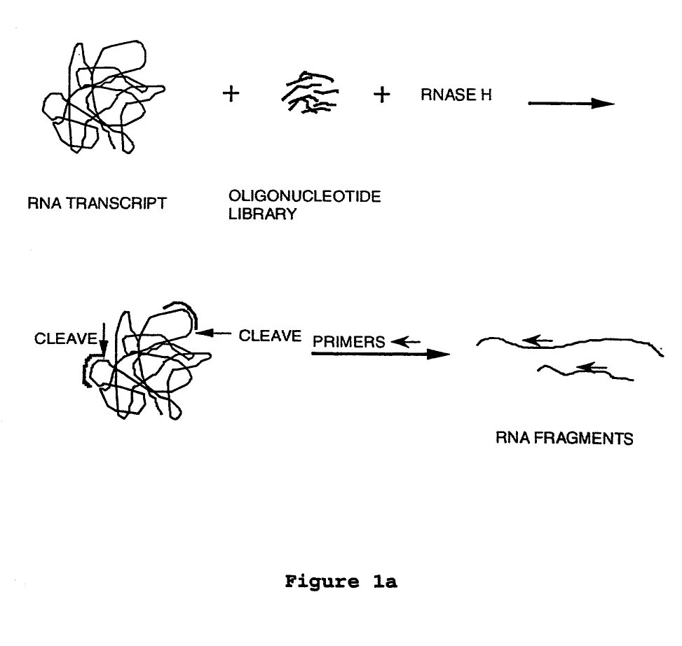

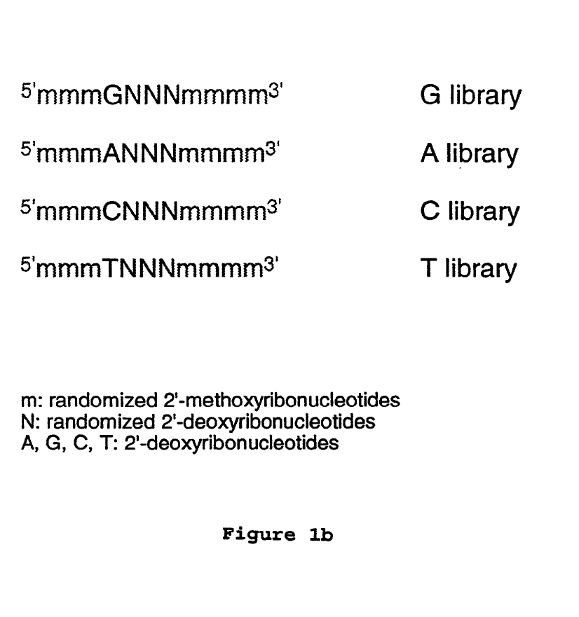

The selection of active antisense sequences has largely been empirical and rather time-consuming. A method was therefore devised for locating sites on an mRNA molecule that are most accessible to hybridization with antisense oligonucleotides (Ho et al., 1996; Ho et al., 1998). This was accomplished (FIG. 1a) by probing an RNA transcript with a library of chemically synthesized, semi-random oligonucleotides (FIG. 1b). When mixed together, the accessible regions of the RNA should hybridize with complementary sequences found within the library. These regions are subsequently identified using ribonuclease H (RNase H), which catalyzes the hydrolytic cleavage of the phosphodiester backbone of only the RNA strand of a hybrid RNA-DNA duplex. Sequencing of the RNA fragments produced should allow identification of those regions in a particular mRNA sequence which can then serve as sites for targeting antisense oligonucleotides. Application of this RNA-mapping method to the RNA transcript containing the entire coding region of the CRF₂ receptor mRNA led to the identification of multiple RNA sites which are accessible to hybridization with antisense oligonucleotides (Table 1).

TABLE 1

| ACCESSIBLE SITE | LOCATION |
|---|---|
| A | 315-338 |
| B | 417-455 |
| C | 608-625 |
| D | 677-731 |
| E | 763-813 |
| F | 859-882 |
| G | 911-941 |
| H | 1018-1031 |
| I | 1161-1185 |
| J | 1238-1258 |
| K | 1385-1417 |

Table 1: Sites in the CRF₂ receptor mRNA that are accessible to oligonucleotide hybridization. Sequence information is with reference to RNU16253.GB_RO (GenBank sequence, accession number U16253).

Antisense oligonucleotides 15 to 25 nucleotides in length can be designed by targeting the 5′-end of the antisense oligonucleotide to accessible sites defined by the data provided in Table 1. For example, the antisense oligonucleotide used in the studies described below was a 20 nucleotide sequence (TGA CGC AGC GGC ACC AGA CC) targeted to positions 758-777 of accessible site E.

Antisense sequences directed against several of these sites inhibited CRF₂ receptor synthesis by at least 50% in cell-based assays. This was determined through a CRF₂ radioligand-binding assay using ¹²⁵I-sauvagine. The antisense inhibition was sequence specific as 4-base mismatches of the antisense oligonucleotides produced only minimal reductions in ¹²⁵I-sauvagine binding. In addition, these sequences also suppressed CRF₂ receptor synthesis in vivo.

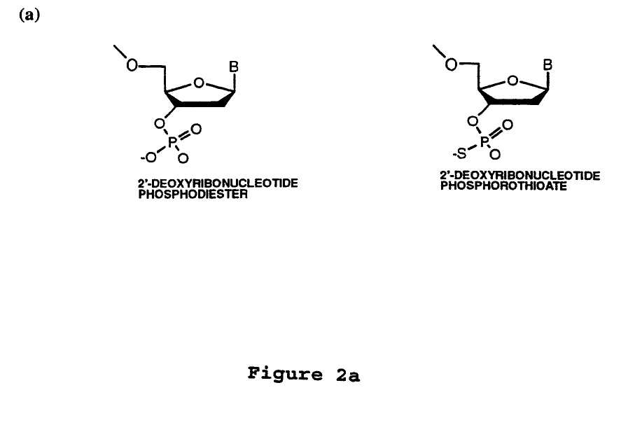

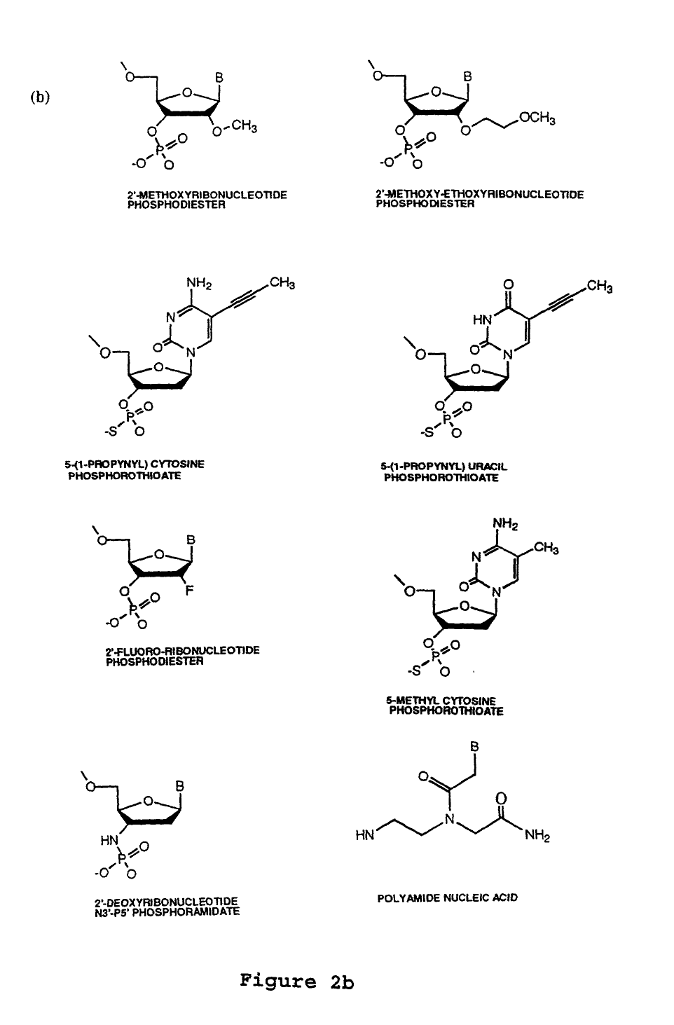

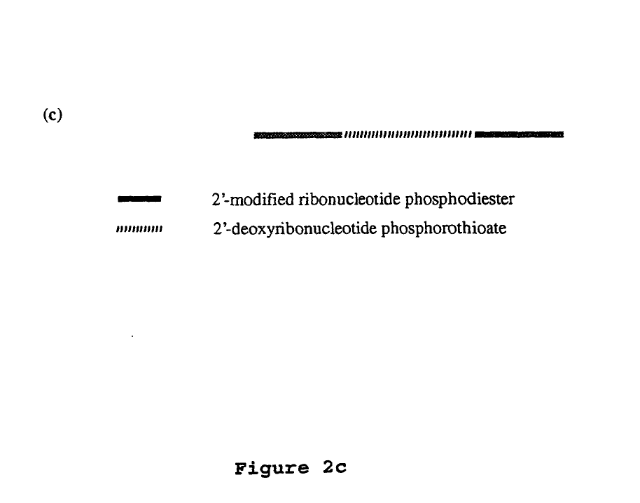

The two chemical versions of oligonucleotides most commonly used in CNS in vivo antisense experiments are 2′-deoxyribonucleotide phosphodiester oligonucleotides and 2′-deoxyribonucleotide phosphorothioate oligonucleotides (FIG. 2a). While being identical in chemical structure to double stranded DNA in genes, single stranded phosphodiester oligonucleotides however are susceptible to exonucleolytic and endonucleolytic degradation, with a half-life in serum of 20 minutes. Even in the ‘privileged’ environment of the brain with its lower level of nuclease activity, phosphodiester oligonucleotides are degraded, albeit more slowly. Phosphorothioate oligonucleotides, where one of the non-bridging phosphate oxygen molecules is replaced with a sulfur, are far more resistant to degrading enzymes. In serum and in tissue culture experiments, phosphorothioate oligonucleotides have a half-life of over 12 hours and analysis of phosphorothioates extracted from rat brain shows these oligonucleotides to be chemically intact for at least 24 hours. However, administration of these oligonucleotides in the brain produces chemistry-related but not sequence-specific toxic effects. Febrile responses, induction of inflammatory mediators, weight loss and various clinical signs have recently been reported. In our experiments, CRF₂ antisense sequences containing the phosphorothioate chemistry produced large inhibitory effects on the CRF₂ receptor but caused significant weight loss (similar to the Heinrichs report) and a host of pathophysiological symptoms in the treated animals. These effects were observed with many different sequences, antisense as well as control sequences, precluding the possibility that they are target-related effects.

Strategies that reduced the overall phosphorothioate content in these oligonucleotides were the most effective at maintaining oligonucleotide potency while circumventing these toxic effects. Chimeric oligonucleotides where up to 60% of the 2′-deoxyribonucleotide phosphorothioate residues were replaced with modified ribonucleotide phosphodiester residues (see FIG. 2b) eliminated weight loss and all other signs of toxicity. The remaining 40% of 2′-deoxyribonucleotide phosphorothioate residues are present in a contiguous stretch to facilitate RNase H cleavage of the targeted mRNA species (FIG. 2c). Incorporation of other chemical analogs such as 5-propynyl-2′-deoxycytidine, 5-propynyl-2′-deoxyuridine and 5-methyl-2′-deoxycytidine (but with phosphorothioate linkages, FIG. 2b) also significantly reduced these toxic effects. In addition to having reduced toxicity, these modified nucleotide residues are more resistant to cellular nuclease degradation than 2′-deoxyribonucleotide phosphodiester residues.

The absence of functional changes resulting from small antisense inhibitory effects often leads to non-interpretable results. This is due to the uncertainty of whether the experiment produced truly negative results or whether the antisense inhibition was insufficient to reveal a functional change. In addition to the antisense sequence, the magnitude of antisense inhibitory effects is influenced by the duration of antisense treatment and its relation to the half-life of the targeted protein. While the half-life of the CRF₂ receptor is unknown, half-lives of other 7-transmembrane receptors in rodent brain (of which the CRF₂ receptor is a member) are on the order of 2-3 days. Maximal inhibitory effects are typically seen after antisense treatment for at least 3 protein half-lives. While CRF₂ antisense administration for 5 days produced a 40-50% inhibition of the receptor, increasing the duration of dosing to 9 days led to a 70-80% inhibitory effect on receptor binding. In addition, quantitative in situ hybridization revealed comparable decreases in CRF₂ receptor mRNA. The 4-base mismatch control oligonucleotide produced minimal decreases in both receptor and mRNA binding under these conditions. Therefore, in contrast to Heinrichs et al. whose CRF₂ antisense oligonucleotide produced only a 15-20% CRF₂ receptor reduction concomitant with significant weight loss in the treated animals, we have optimized antisense reagents for the study of CRF₂ receptor function. Antisense sequence selection using the RNA mapping method, combined with optimized nucleotide chemistries resulted in potent antisense sequences, which when administered in rodents for 8-10 days, produced large (around 70%) decreases in CRF₂ receptor binding.

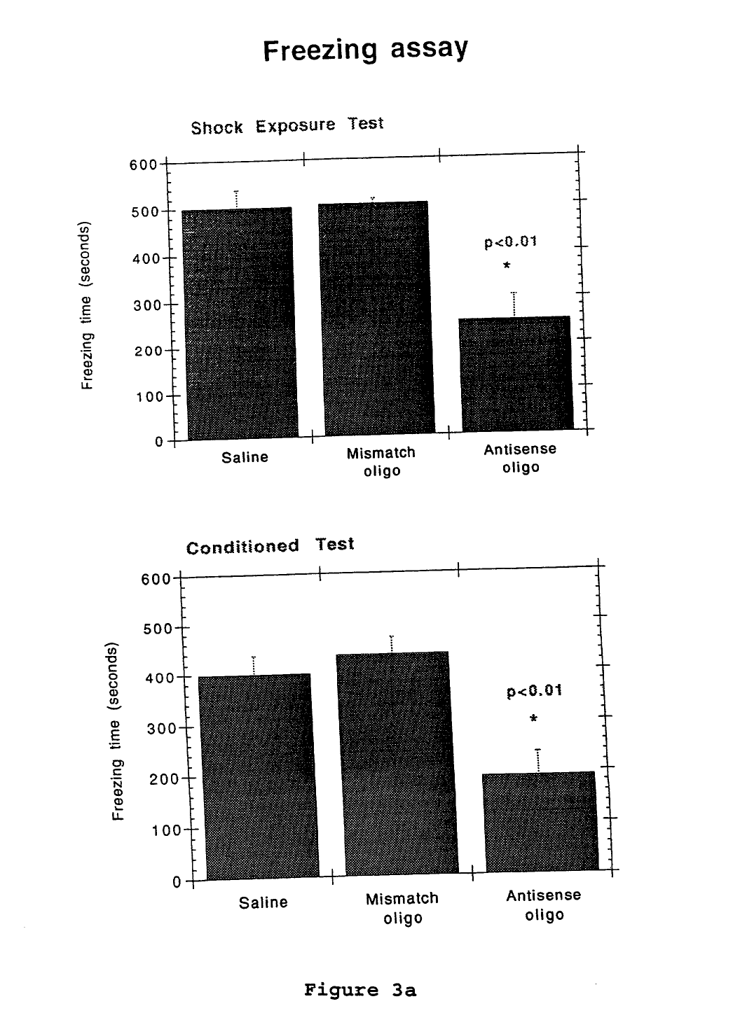

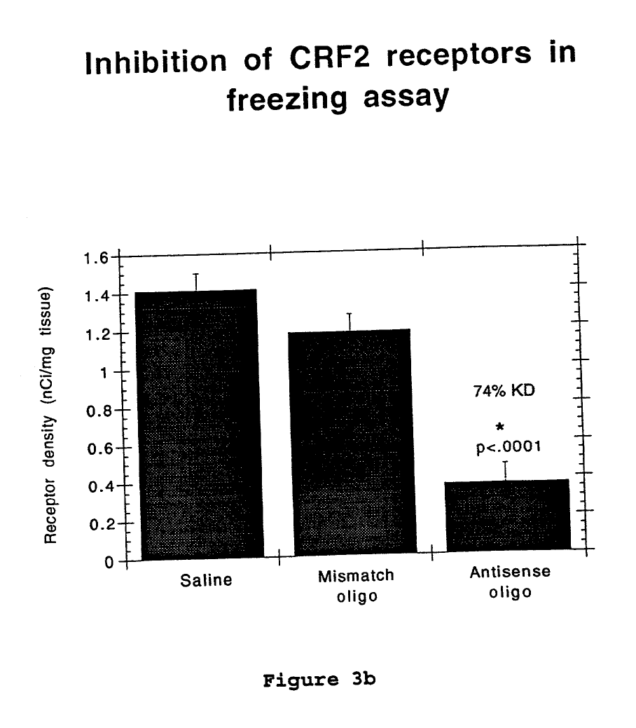

CRF₂ antisense oligonucleotides were administered intracerebroventricularly to target the lateral septum, a brain region containing high levels of CRF₂ receptor and mRNA. The lateral septum is part of the limbic brain region known for its involvement in modulating fear and emotion. Rats treated with saline, antisense and mismatch-control oligonucleotides were tested in two different behavioral models of anxiety. Rodents display a characteristic freezing behavior when experiencing fear and anxiety. In the freezing model of anxiety, such behavior is induced by exposure to brief electrical foot-shocks. When such rats are returned to the shock box after several intervening days, they exhibit freezing behavior even in the absence of further shock exposure. Administration of anxiolytic drugs such as benzodiazepines and selective serotonin reuptake inhibitors reduces the duration of freezing when previously shocked animals are returned to the shock box. In the antisense experiments, dosing of oligonucleotides began after two consecutive days of foot-shock treatment. Two hours following the last oligonucleotide administration on day 8 of dosing, rats were returned to the shock box and observed for 10 minutes. In this part of the experiment, which examines the effect of the pharmacological agent on conditioned fears, the antisense oligonucleotide, but not its mismatch control, reduced the duration of freezing by 50% (FIG. 3a). Following this initial 10 minute period, the rats received two brief foot-shocks and were observed for an additional 10 minutes. Again, the antisense-treated rats exhibited a 50% reduction in the duration of freezing compared to saline, or mismatch oligonucleotide-treated animals (FIG. 3a). These data constitute the first demonstration of function in CRF₂ receptors. Receptor autoradiographic analysis of the septal brain region in these rats showed a 70% reduction in ¹²⁵I-sauvagine binding to CRF₂ receptors in the antisense treated rats (FIG. 3b). Therefore, inhibition of CRF₂ receptors leads to reduced anxiety levels, indicating that the anxiogenic effects of the CRF peptide are mediated not only through CRF₁ receptors but also by CRF₂ receptors. Furthermore, a robust suppression of CRF₂ receptors produced important functional consequences that may not be apparent at lower levels of CRF₂ receptor inhibition. These results implicate the CRF₂ receptor in modulating fear and anxiety responses.

The elevated plus maze (EPM) is widely used for the determination of anxiolytic or anxiogenic drug effects. The apparatus consists of a +-shaped maze, elevated 50 cm above the floor. Two opposing arms are open and exposed to the environment while the other two arms are enclosed with black Plexiglas sides. In rodents, exposure to the EPM produces an approach/avoidance conflict which generally causes the animal to spend most of its time in the closed arms of the maze. Such approach/avoidance conflicts are thought to be important components underlying the occurrence of some types of human anxiety disorders. Importantly, drugs currently prescribed for the treatment of anxiety disorders are effective in producing anxiolytic responses in rodents tested in the EPM.

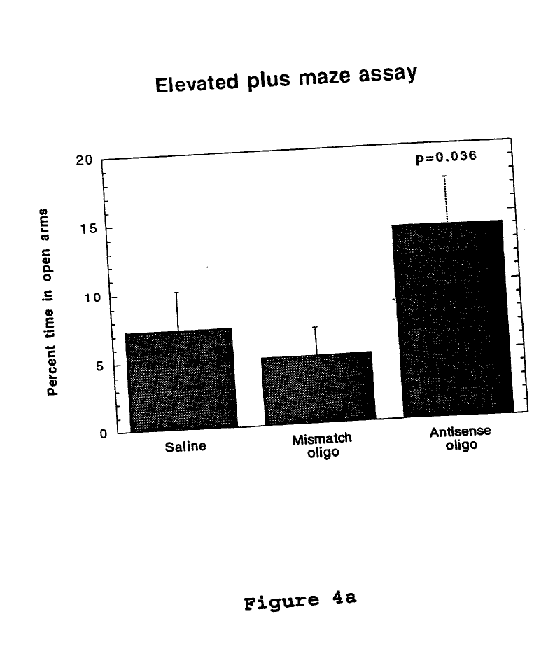

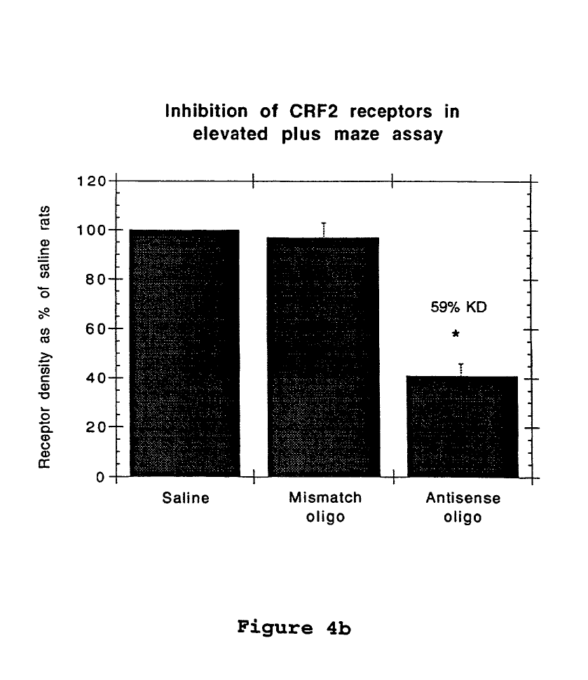

In the antisense experiment, rats were dosed for 8 days and then tested in the EPM 2 hours after the last oligonucleotide injection. Rats treated with the antisense oligonucleotide spent significantly more time in the open, exposed arms of the maze (FIG. 4a). Such behavior is indicative of a reduced state of anxiety. Mismatch oligonucleotide-treated rats were not statistically different from saline-treated rats. Binding of ¹²⁵I-sauvagine to CRF₂ receptors in the lateral septum was reduced by 60% by the antisense oligonucleotide in this experiment (FIG. 4b).

Analysis of the sum of entries into open and closed arms of the maze revealed no differences between the three treatment groups (data not shown). In addition, in the locomotor activity test, all three treatment groups were again indistinguishable (data not shown). Taken together, these data show that the motor function of the rats was not significantly altered by oligonucleotide treatment.

It has been demonstrated that antisense inhibition of 7-transmembrane receptor systems produces physiological effects that are similar to those obtained through receptor blockade by selective small molecule antagonists (Ho et al., 1998). Our CRF₂ antisense results therefore imply that in addition to antisense suppression of CRF₂ receptors, blockade of this receptor by small molecule ligands should also result in anxiolytic effects. Therefore, small molecule or peptide antagonists of CRF₂ receptors should be effective anxiolytic agents with beneficial therapeutic value.

The term “Pharmaceutically acceptable prodrugs” as used herein means those prodrugs of the compounds useful according to the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” means compounds that are rapidly transformed in vivo to yield the parent compound, for example by hydrolysis in blood. Functional groups which may be rapidly transformed, by metabolic cleavage, in vivo form a class of groups reactive with the carboxyl group of the compounds of this invention. They include, but are not limited to such groups as alkanoyl (such as acetyl, propionyl, butyryl, and the like), unsubstituted and substituted aroyl (such as benzoyl and substituted benzoyl), alkoxycarbonyl (such as ethoxycarbonyl), trialkylsilyl (such as trimethyl- and triethysilyl), monoesters formed with dicarboxylic acids (such as succinyl), and the like. Because of the ease with which the metabolically cleavable groups of the compounds useful according to this invention are cleaved in vivo, the compounds bearing such groups act as pro-drugs. The compounds bearing the metabolically cleavable groups have the advantage that they may exhibit improved bioavailability as a result of enhanced solubility and/or rate of absorption conferred upon the parent compound by virtue of the presence of the metabolically cleavable group. A thorough discussion of prodrugs is provided in the following: Design of Prodrugs, H. Bundgaard, ed., Elsevier, 1985; Methods in Enzymology, K. Widder et al; Ed., Academic Press, 42, p. 309-396, 1985; A Textbook of Drug Design and Development, Krogsgaard-Larsen and H. Bundgaard, ed., Chapter 5; “Design and Applications of Prodrugs” p. 113-191, 1991; Advanced Drug Delivery Reviews, H. Bundgard, 8, p. 1-38, 1992; Journal of Pharmaceutical Sciences, 77, p. 285, 1988; Chem. Pharm. Bull., N. Nakeya et al; 32, p. 692, 1984; Pro-drugs as Novel Delivery Systems, T. Higuchi and V. Stella, Vol. 14 of the A.C.S. Symposium Series, and Bioreversible Carriers in Drug Design, Edward B. Roche, ed., American Pharmaceutical Association and Pergamon Press, 1987, which are incorporated herein by reference.

The term “Pharmaceutically acceptable salts” means the relatively non-toxic, inorganic and organic acid addition salts, and base addition salts, of compounds of the present invention. These salts can be prepared in situ during the final isolation and purification of the compounds. In particular, acid addition salts can be prepared by separately reacting the purified compound in its free base form with a suitable organic or inorganic acid and isolating the salt thus formed. Exemplary acid addition salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, sulphamates, malonates, salicylates, propionates, miethylene-bis-b-hydroxynaphthoates, gentisates, isethionates, di-p-toluoyltartrates, methane-sulphonates, ethanesulphonates, benzenesulphonates, p-toluenesulphonates, cyclohexylsulphamates and quinateslaurylsulphonate salts, and the like. (See, for example S. M. Berge, et al., “Pharmaceutical Salts,” J. Pharm. Sci., 66: p. 1-19 (1977) which is incorporated herein by reference.) Base addition salts can also be prepared by separately reacting the purified compound in its acid form with a suitable organic or inorganic base and isolating the salt thus formed. Base addition salts include pharmaceutically acceptable metal and amine salts. Suitable metal salts include the sodium, potassium, calcium, barium, zinc, magnesium, and aluminum salts. The sodium and potassium salts are preferred. Suitable inorganic base addition salts are prepared from metal bases which include sodium hydride, sodium hydroxide, potassium hydroxide, calcium hydroxide, aluminium hydroxide, lithium hydroxide, magnesium hydroxide, zinc hydroxide. Suitable amine base addition salts are prepared from amines which have sufficient basicity to form a stable salt, and preferably include those amines which are frequently used in medicinal chemistry because of their low toxicity and acceptability for medical use. ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine, and dicyclohexylamine, and the like.

The term “CRF₂ antisense oligonucleotides”, as used herein, refers to short oligonucleotides (typically from about 15 to about 25 nucleotides in length) which are designed to be complementary to a portion of an mRNA of the CRF₂ receptor. Hybridization of an antisense oligonucleotide to its mRNA target site through Watson-Crick base-pairing initiates a cascade of events which terminate in oligonucleotide-directed degradation of the targeted mRNA of the CRF₂ receptor.

The term “CRF₂ receptor(s)”, as used herein, refers to cell surface receptors as described in U.S. Pat. No. 5,786,203, issued Jul. 28, 1998, the contents of which are herein incorporated by reference.

The term “defined accessible site”, as used herein, refers to multiple sites in the CRF₂ receptor mRNA which are accessible to hybridization with antisense oligonucleotides. These sites are further delineated in Table 1 above.

The term “modified nucleotide residue”, as used herein, includes but is not limited to 2′-methoxyribonucleotide phosphodiesters, 2′-methoxy-ethoxyribonucleotide phosphodiesters, 2′-fluoro-ribonucleotide phosphodiesters, 5-(1-propynyl)cytosine phosphorothioate, 5-(1-propynyl)uracil phosphorothioate, 5-methyl cytosine phosphorothioate, 2′-deoxyribonucleotide-N3′-P5′phosphoramidate, polyamide nucleic acids, and locked nucleic acids having the formula:

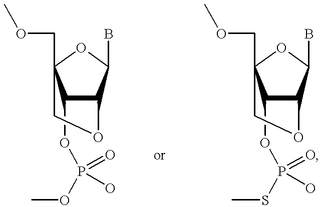

wherein B is a purine or pyimidine base.

An embodiment of the invention provides a method of treating psychiatric disorders including, but not limited to, anxiety, obsessive-compulsive disorder, panic disorders, post-traumatic stress disorder, phobias, anorexia nervosa and depression in a patient, by administering to the patient requiring such treatment a therapeutically effective amount of a pharmaceutical composition comprising antisense oligonucleotides comprised of chimeric oligonucleotides where 10-70% of the 2′-deoxyribonucleotide phosphorothioate residues are replaced with modified nucleotide residues.

A preferred embodiment provides that the modified nucleotide residues of the antisense oligonucleotides are selected from the following group: 2′-methoxyribonucleotide phosphodiesters, 2′-methoxy-ethoxyribonucleotide phosphodiesters, 2′-fluoro-ribonucleotide phosphodiesters, 5-(1-propynyl)cytosine phosphorothioate, 5-(1-propynyl)uracil phosphorothioate, 5-methyl cytosine phosphorothioate, 2′-deoxyribonucleotide-N3′-P5′phosphoramidate, and polyamide nucleic acids.

A more preferred embodiment provides the antisense oligonucleotide is from about 15 to about 25 nucleotides in length.

Another embodiment provides a method of treating a patient having a disease mediated by a CRF receptor protein, comprising:

(a) designing a chimeric antisense oligonucleotide specific for the CRF receptor mRNA;

(b) determining a composition that mimics the biological effect of the antisense oligonucleotide; and

(c) administering to the patient the composition that inhibits binding of the endogenous ligand to its CRF receptor.

Another embodiment provides a method of treating a patient having a disease mediated by a CRF receptor protein, comprising:

(a) designing a chimeric antisense oligonucleotide specific for the CRF receptor mRNA;

(b) determining a composition that mimics the biological effect of the antisense oligonucleotide; and

(c) administering to the patient a composition that mimics the action of the endogenous ligand at the CRF receptor.

Another embodiment of the present invention provides a method for treating a patient having a disease mediated by CRF, comprising administering to the patient a composition that effectively inhibits binding of CRF, or other closely related peptides, to the CRF₂ receptor.

Another embodiment of the present invention provides a method of designing an inhibitor of the CRF₂ receptor comprising the steps of determining the three-dimensional structure of such receptor, analyzing the three-dimensional structure for the likely binding sites of substrates, synthesizing a molecule that incorporates a predictive reactive site, and determining the receptor-inhibiting activity of the molecule.

Another embodiment of the present invention provides sequences of antisense oligonucleotides composed of chimeric oligonucleotides where between 10-70% of the 2′-deoxyribonucleotide phosphorothioate residues are replaced with modified nucleotide residues.

A more preferred embodiment of the present invention provides sequences of antisense oligonucleotides composed of chimeric oligonucleotides where between 15-70% of the 2′-deoxyribonucleotide phosphorothioate residues are replaced with modified nucleotide residues.

A more preferred embodiment of the present invention provides sequences of antisense oligonucleotides composed of chimeric oligonucleotides where between 20-70% of the 2′-deoxyribonucleotide phosphorothioate residues are replaced with modified nucleotide residues.

A more preferred embodiment of the present invention provides sequences of antisense oligonucleotides composed of chimeric oligonucleotides where between 25-70% of the 2′-deoxyribonucleotide phosphorothioate residues are replaced with modified nucleotide residues.

A more preferred embodiment of the present invention provides sequences of antisense oligonucleotides composed of chimeric oligonucleotides where between 30-70% of the 2′-deoxyribonucleotide phosphorothioate residues are replaced with modified nucleotide residues.

A more preferred embodiment of the present invention provides sequences of antisense oligonucleotides composed of chimeric oligonucleotides where between 35-70% of the 2′-deoxyribonucleotide phosphorothioate residues are replaced with modified nucleotide residues.

A more preferred embodiment of the present invention provides sequences of antisense oligonucleotides composed of chimeric oligonucleotides where between 40-70% of the 2′-deoxyribonucleotide phosphorothioate residues are replaced with modified nucleotide residues.

A more preferred embodiment of the present invention provides sequences of antisense oligonucleotides composed of chimeric oligonucleotides where between 45-70% of the 2′-deoxyribonucleotide phosphorothioate residues are replaced with modified nucleotide residues.

A more preferred embodiment of the present invention provides sequences of antisense oligonucleotides composed of chimeric oligonucleotides where between 50-70% of the 2′-deoxyribonucleotide phosphorothioate residues are replaced with modified nucleotide residues.

A more preferred embodiment of the present invention provides sequences of antisense oligonucleotides composed of chimeric oligonucleotides where between 55-70% of the 2′-deoxyribonucleotide phosphorothioate residues are replaced with modified nucleotide residues.

An even more preferred embodiment of the present invention provides sequences of antisense oligonucleotides composed of chimeric oligonucleotides where between 60-70% of the 2′-deoxyribonucleotide phosphorothioate residues are replaced with modified nucleotide residues.

A further preferred embodiment of the present invention provides for antisense oligonucleotides having a target base located within a defined accessible site, having a starting point at any base located within the defined accessible site, and having a length from about 15 to about 25 bases.

A most preferred embodiment of the present invention provides for antisense oligonucleotides comprising the following sequences:

(a) TGT ACG TGT TGC GCA AGA GG;

(b) GGT GGG CGA TGT GGG AAT G;

(c) GGA TGA AGG TGG TGA TGA GG; and

(d) TGA CGC AGC GGC ACC AGA CC.

Another embodiment of the present invention provides a screening assay for determining compounds useful in the treatment of psychiatric disorders including, but not limited to, anxiety, obsessive-compulsive disorder, panic disorders, post-traumatic stress disorder, phobias and depression utilizing antisense oligonucleotides.

Another embodiment of the present invention provides a method of determining the structure of the binding region of the CRF₂ receptor.

Administration of a CRF₁ receptor ligand in combination with a CRF₂ receptor ligand, may afford an efficacy advantage over the CRF₁ receptor ligand and CRF₂ receptor ligand alone, and may do so while permitting the use of lower doses of each. A lower dosage minimizes the potential of side effects, thereby providing an increased margin of safety. The combination of a compound of the present invention with such additional therapeutic agents is preferably a synergistic combination. Synergy, as described for example by Chou and Talalay, Adv. Enzyme Regul. 22:27-55 (1984), occurs when the therapeutic effect of the compound and agent when administered in combination is greater than the additive effect of the either the CRF₁ receptor ligand and CRF₂ receptor ligand when administered alone. In general, a synergistic effect is most clearly demonstrated at levels that are (therapeutically) sub-optimal for either the CRF₁ receptor ligand or CRF₂ receptor ligand alone, but which are highly efficacious in combination.

CRF₁ receptor antagonists are active in several animals models of anxiety (Lundkvist, J., Chai, Z., Teheranian, R., Hasanvan, H., Bartfai, T., Jenck, F., Widmer, U. & Moreau, J. -L. (1996) Eur. J. Pharmacol. 309, 195-200; and Weninger, S. C., Dunn, A. J., Muglia, L. J., Dikkes, P., Miczek, K. A., Swiergiel, A. H., Berridge, C. W. & Majzoub, J. A. (1999) Proc. Natl. Acad. Sci. USA 96, 8283-8288). DPC904 (Gilligan, P. J., Baldauf, C., Cocuzza, A., Chidester, D., Zaczek, R., Fitzgerald, L., McElroy, J., Smith, M. A., Shen, H. -S. L., Saye, J. A., Christ, D., Trainor, G. L., Robertson, D. W. & Hartig, P. R. (2000) Bioorganic Med. Chem. 8, 181-189, 2000), a highly selective and potent pyrazolo-pyrimidine antagonist of the CRF₁ receptor, was tested in the conditioned anxiety test and found a dose-dependent reduction in freezing duration (FIG. 7a). Because central CRF₁ and CRF₂ receptors do not overlap significantly in their anatomical distribution (Chalmers, D. T., Lovenberg, T. W. & De Souza, E. B. (1995) J. Neuroscience 15, 6340-6350; and Rominger, D. H., Rominger, C. M., Fitzgerald, L. W., Grzanna, R., Largent, B. L. & Zaczek, R. (1998) J. Pharmacol. Exp. Ther. 286, 459-468), a study was designed to determine whether simultaneous inhibition of both receptor subtypes would produce more potent reductions in freezing. Animals were dosed intracerebroventricularly for seven days with either saline or antisense oligonucleotide. Twenty four hours after the last icv dose, rats received an oral administration of either vehicle (methocel) or DPC904. Animals that received either DPC904 or the antisense oligonucleotide alone exhibited significant reductions in freezing as previously observed. In animals which received both DPC904 and the antisense oligonucleotide, freezing was reduced significantly below the level of DPC904-treated or antisense-treated animals in the conditioned anxiety test (FIG. 7b). Although acute treatment with DPC904 reduced freezing duration in the shock re-exposure test, simultaneous inhibition of both receptors did not produce effects that were different from that obtained with the CRF₂ antisense oligonucleotide alone (FIG. 7b). CRF₂ receptor binding was reduced to similar extents in both the antisense-treated groups of animals (Saline/methocel: 1.20±0.05 nCi/mg, Saline/DPC904: 1.21±0.05 nCi/mg, antisense oligonucleotide/methocel: 0.51±0.08 nCi/mg, antisense oligonucleotide/DPC904: 0.45±0.04 nCi/mg; p<0.001 for both antisense groups vs non-oligonucleotide-treated groups).

It is to be understood that this invention covers all appropriate combinations of the particular and preferred groupings or embodiments referred to herein.

The invention can be further understood by the following examples in which parts and percentages are by weight unless otherwise indicated.

### EXAMPLE 1

### Synthesis and Purification of Oligonucleotides for in Vivo Experiments

Oligonucleotides were synthesized on an automated ABI 394 RNA/DNA synthesizer using standard synthesis protocols. The antisense and mismatch oligonucleotides used in experiments described in FIGS. 3 and 4 consist of the following sequences:

Antisense: TGA CGC agc ggc acC AGA CC

Mismatch: TGA GGC acc gga acC ACA CC

where upper case letters denote 2′-methoxyribonucleotide phosphodiester residues, and lower case letters denote 2′-deoxyribonucleotide phosphorothioate residues. 2′-methoxyribonucleotide phosphoramidites were purchased from Chem Genes, propynyl and 5-methyl cytidine phosphoramidites were obtained from Glen Research and 2′-fluorophosphoramidites were from NeXstar. Beaucage reagent for the synthesis of phosphorothioate linkages and fluorescein phosphoramidite for 5′-labeling of oligonucleotides was purchased from Glen Research. These reagents were used according to manufacturer's instructions.

Crude oligonucleotide mixtures were purified by reverse phase HPLC on a PRP-3 column (Hamilton Co.) using a gradient of acetonitrile and 0.1 M aqueous triethylammonium acetate. Fractions collected off the HPLC column were lyophilized twice to remove excess triethylammonium acetate. An aqueous solution of the oligonucleotide was then extracted several times with butanol. Cation exchange was accomplished using ethanol precipitation in the presence of 0.3 M sodium acetate. The pH of the oligonucleotide solution was then brought up to pH 7.0 by addition of 0.01 M sodium hydroxide. The oligonucleotide was further purified by size exclusion chromatography using NAP-25 columns (Pharmacia) to remove residual fluorescein phosphoramidite reagent. Sterilization was accomplished by filtration through a 0.2 □m cellulose acetate filter (Rainin) and quantitated by UV spectrometry. The purity of oligonucleotides was determined by capillary gel electrophoresis (PACE2100, Beckman Instruments). Stocks of oligonucleotide in distilled water were stored at −20° C.

### EXAMPLE 2

### Animals and Surgery

Male Sprague Dawley rats (Charles River) weighing 320-360 g at the time of surgery, were individually housed in stainless steel cages and provided free access to food and water. Following a 4 day adaptation period, rats were stereotaxically implanted bilaterally, under Rompun (100 mg/kg) and ketamine (9 mg/kg) anesthesia, with chronic 26-gauge guide cannulae aimed at the lateral ventricles. Stereotaxic co-ordinates were: incisor bar 3.3 mm below interaural line; 0.2 mm posterior to bregma; ±2.7 mm lateral to midline; 3.8 mm ventral to skull surface and a 24° angle. The injector (33 gauge) projected beyond the tip of the guide cannulae by 0.5 mm. The animals were adapted by daily handling beginning 2 days after surgery.

All animal care and use procedures described were approved by the Institutional Animal Care and Use Committee (IACUC). DuPont Pharmaceuticals Research Laboratories is accredited by the Association for the Assessment and Accreditation of Laboratory Animal Care (AAALAC International).

### EXAMPLE 3

### Oligonucleotide Administration

Oligonucleotide infusions were started on the 8th day following surgery when rats were about 20 g above surgery weights. Fresh oligonucleotide solutions were prepared daily by dissolving lyophilized oligonucleotide pellets in sterile saline. Rats were weighed daily at 9:00 AM before oligonucleotide infusion. Using a microprocessor controlled syringe pump (Stoelting), 1 □L of solution was injected per ventricle over 2 minutes. The injector was left in the guide cannula for an additional minute. Separate injectors for each individual rat were rinsed with ethanol and sterile water, and dried between daily injections.

### EXAMPLE 4

### Freezing Assay of Anxiety

The shock box consisted of a black Plexiglas chamber with walls and cover. The doors of the box were constructed of clear Plexiglas over which one-way mirrors were attached for observation. The floor of the box contained a Coulbourn stainless steel shock grid with the bars of the grid spaced 1 cm apart. On the 8th day following surgical implantation of the guide cannulae, rats were placed in the box and allowed to habituate for 2 minutes. A total of 3 scrambled, randomized non-escapable foot-shocks (1.0 mA, 1 second duration) were then delivered at 20 second intervals to the grid floor. The rat was observed for freezing behavior for 15 minutes before it was returned to its home cage.

Oligonucleotide treatment was initiated the day following shock treatment. Animals were dosed for seven consecutive days. Twenty four hours after the rats were returned to the shock box and observed for freezing behavior for 10 minutes. This was followed by the administration of 2 foot-shocks (1.0 mA, 1 second duration, 20 second interval) after which the rat was observed for freezing for another 10 minutes. Immediately following this last 10 minute period, the rat was euthanitized.

### EXAMPLE 5

### Elevated Plus Maze Assay

Oligonucleotide treatment of rats was begun on the 8th day following surgery. Rats were tested in the EPM 2 hours following dosing on the 8th day of treatment. At the start of the test, the rat was placed in the center square of the maze and its exploratory behavior during the ensuing 10 minutes was recorded by video-camera. An observer situated outside the test room scored the time spent in the open and closed arms, as well as the number of entries into each arm of the maze. The rats were euthanitized immediately following the conclusion of the test.

### EXAMPLE 6

### Tissue Preparation

Rats were sacrificed by exposure to CO₂. Brains were removed and frozen in methylbutane cooled on dry ice before storage at −80° C. Twenty μm sections through the lateral septum were cut on a cryostat(Kopf Instruments) for receptor autoradiography.

### EXAMPLE 7

### CRF₂ Receptor Autoradiography

After warming to room temperature for 1 hour, brain sections were preincubated for 5 minutes in 50 mM Tris-HCL (pH 7.5) containing 10 mM MgCl₂, 2 mM EGTA (ethylene glycol-bis(p-aminoethyl ether) N,N,N′,N′-tetraacetic acid), 0.1% ovalbumin, 0.08 TIU aprotinin and 0.1 mM bacitracin. Total binding was defined using 0.15 nM ¹²⁵I-sauvagine (New England Nuclear). CRF₂ specific binding was determined in the presence of 1 μM SC-241, a CRF₁ selective receptor antagonist (D. H. Rominger et alJ. Pharmacol. Exp. Therap., 286, 459-468, 1998). Non-specific binding was determined using 1 μM a-helical CRF (American Peptide). Incubations were performed in preincubation buffer containing radioligand and appropriate antagonists for 150 minutes. Tissue sections were then washed twice for 5 minutes each, in PBS containing 0.01% Triton X-100. After a final water rinse, excess water was aspirated and the sections were air-dried overnight. The sections and ¹²⁵I standard strips (Amersham) were exposed to Hyperfilm μ-max (Amersham) for 72 hours.

Quantitation of CRF2 specific binding was performed using the NIH ImageMG 1.44 program. Optical density readings were converted to nCi of ligand bound per mg of protein tissue using ¹²⁵I standard strips. Between 7 to 9 adjacent sections were quantitated per rat.

### EXAMPLE 8

### Combination Treatment with CRF1 Receptor Antagonist and CRF2 Antisense Oligonucleotide

Thirty two to forty rats were subjected to conditioning foot-shock treatments as described in Example 4 (first paragraph). Following foot-shock, the animals were equally divided into 2 groups. The first group received intracerebroventricular saline injections for 7 consecutive days, while the second group of animals received intracerebroventricular injections of the antisense oligonucleotide (2.5 nmol in each lateral ventricle) for 7 consecutive days. On the eighth day, each group of animals was further subdivided into 2 groups. Half of the saline-treated animals received DPC 904 (in methocel) at a dose of 10 mg/kg P.O. (designated the S/R1 group). The other half of the saline animals received the vehicle methocel (designated the S/M group). Rats dosed with the antisense oligonucleotide were similarly treated, i.e. half of those animals received DPC 904 (in methocel) at a dose of 10 mg/kg p.o. (designated the R2/R1 group). The other half of the antisense-treated animals received the vehicle methocel (designated the R2/M group). Thirty minutes following oral dosing, animals were tested in the shock box as described in Example 4 (second paragraph).

The present invention may be embodied in other specific forms without departing from the spirit or essential attributes thereof.

What is claimed is:

1. A method of treating a disorder associated with CRF₁ and CRF₂ receptor activity, comprising administering to a patient in need thereof a therapeutically effective amount of a CRF₁ receptor ligand and a CRF₂ receptor ligand, or pharmaceutically acceptable salts or prodrugs thereof.

2. A method according to claim 1, wherein the CRF₁ receptor ligand is agonistic of the CRF₁ receptor.

3. A method according to claim 1, wherein the CRF₁ receptor ligand is antagonistic of the CRF₁ receptor.

4. A method according to claim 1, wherein the CRF₂ receptor ligand is agonistic of the CRF₂ receptor.

5. A method according to claim 1, wherein the CRF₂ receptor ligand is antagonistic of the CRF₂ receptor.

6. A method of treating a disorder associated with CRF₁ and CRF₂ receptor activity, comprising administering to a patient in need thereof a therapeutically effective amount of a CRF₁ receptor ligand and a CRF₂ receptor antisense oligonucleotide, or pharmaceutically acceptable salts or prodrugs thereof, wherein the CRF₂ ligand receptor is an antisense oligonucleotides composed of chimeric oligonucleotides wherein between 10-70% of the 2′-deoxyribonucleotide phosphorothioate residues are replaced with modified nucleotide residues.

7. A method according to claim 6, wherein the modified nucleotide residues are selected from the following group: 2′-methoxyribonucleotide phosphodiesters, 2′-methoxy-ethoxyribonucleotide phosphodiesters, 2′-fluoro-ribonucleotide phosphodiesters, 5-(1-propynyl)cytosine phosphorothioate, 5-(1-propynyl)uracil phosphorothioate, 5-methyl cytosine phosphorothioate, 2′-deoxyribonucleotide-N3′-P5′phosphoramidate, polyamide nucleic acids, and locked nucleic acids having the formula: 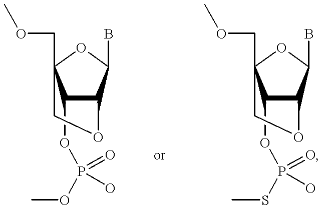
wherein B is a purine or pyimidine base.

8. A method according to claim 6, wherein the oligonucleotide is from about 15 to about 25 nucleotides in length.

9. A method according to claim 6, wherein between 60-70% of the 2′-deoxyribonucleotide phosphorothioate residues of the antisense oligonucleotides are replaced with modified nucleotide residues.

10. A method according to claim 6, wherein the antisense oligonucleotides comprises the following sequences:
(a) TGT ACG TGT TGC GCA AGA GG;
(b) GGT GGG CGA TGT GGG AAT G;
(c) GGA TGA AGG TGG TGA TGA GG; and
(d) TGA CGC AGC GGC ACC AGA CC.

11. A method according to claim 1 or 6, wherein the disorder is a psychiatric disorder.

12. A method according to claim 11, wherein the psychiatric disorder is selected from the group consisting of anxiety, obsessive-compulsive disorder, panic disorders, post-traumatic stress disorder, phobias and depression.

13. A method according to claim 1 or 6, wherein the disorder is selected from the group consisting of head trauma, spinal cord trauma, ischemic neuronal damage (e.g., cerebral ischemia such as cerebral hippocampal ischemia), excitotoxic neuronal damage, epilepsy, stroke, stress induced immune dysfunctions, phobias, muscular spasms, Parkinson's disease, Huntington's disease, urinary incontinence, senile dementia of the Alzheimer's type, multiinfarct dementia, amyotrophic lateral sclerosis, chemical dependencies and addictions (e.g., dependencies on alcohol, cocaine, heroin, benzodiazepines, or other drugs), and hypoglycemia.

14. A method according to claim 1 wherein administering the a CRF₁ receptor ligand and a CRF₂ receptor ligand is concurrent.

15. A method according to claim 1 wherein administering the a CRF₁ receptor ligand and a CRF₂ receptor ligand is sequential.

16. A method of treating a disorder associated with CRF₁ and CRF₂ receptor activity, comprising contacting an effective amount of a CRF₁ receptor ligand and a CRF₂ receptor ligand with a composition containing CRF₁ receptor and CRF₂ receptor.

17. A method of treating a disorder associated with CRF₂ receptor activity, comprising contacting an effective amount of a CRF₂ receptor ligand with a composition containing CRF₂ receptor.

18. A pharmaceutical composition comprising a CRF₁ receptor ligand a CRF₂ receptor ligand, or pharmaceutically acceptable salts or prodrugs thereof, and a pharmaceutical carrier.

19. A pharmaceutical kit for treating or preventing a disorder associated with CRF₁ and CRF₂ receptor activity, said kit comprising a plurality of separate containers, wherein at least one of said containers contains a CRF₁ receptor ligand, or a pharmaceutically acceptable salt or prodrug thereof, and at least another of said containers contains a CRF₂ receptor ligand, or pharmaceutically acceptable salts or prodrugs thereof, and said containers optionally contain a pharmaceutical carrier.

20. A compound having CRF₁ receptor ligand activity and a CRF₂ receptor ligand activity for use in the treatment of psychiatric disorders.

21. A method according to claim 1 wherein the CRF₁ receptor ligand is DPC904 or SC-241.

22. A method according to claim 4 wherein the CRF₂ receptor ligand is sauvagine, urocortin or other CRF₂ peptides.

23. A method according to claim 5 wherein the CRF₂ receptor ligand is anti-sauvagine.

24. A method of enhancing the treatment of a psychiatric disorder comprising administering to a patient in need thereof a therapeutically effective amount of a CRF₁ receptor ligand and a CRF₂ receptor ligand, or pharmaceutically acceptable salts or prodrugs thereof.

25. A method of enhancing the treatment of a psychiatric disorder comprising administering to a patient in need thereof a therapeutically effective amount of a compound having CRF₁ ligand receptor and a CRF₂ ligand receptor activity, or pharmaceutically acceptable salts or prodrugs thereof.

26. A method according to claim 25, wherein the compound is astressin.

27. A method according to claim 6, wherein the antisense oligonucleotide is targeted to regions described in table 1.
